(19) United States
(12) Patent Application Publication
(10) Pub. No.: US 2022/0361823 A1
(43) Pub. Date: Nov. 17, 2022

(54) WEARABLE BLOOD PRESSURE BIOSENSORS, SYSTEMS AND METHODS FOR SHORT-TERM BLOOD PRESSURE PREDICTION

(71) Applicant: INFORMED DATA SYSTEMS INC. d/b/a ONE DROP, New York, NY (US)

(72) Inventors: Daniel R. Goldner, Minnetonka, MN (US); Ydo Wexler, Haifa (IL); Jeffrey Dachis, Brooklyn, NY (US)

(21) Appl. No.: 17/744,446

(22) Filed: May 13, 2022

Related U.S. Application Data
(60) Provisional application No. 63/188,641, filed on May 14, 2021.

(51) Int. Cl. A61B 5/00; A61B 5/0205; A61B 5/145; A61B 5/11

(52) U.S. Cl. CPC A61B 5/7275; A61B 5/486; A61B 5/7267; A61B 5/0205; A61B 5/6802; A61B 5/685; A61B 5/14503; A61B 5/74; A61B 5/0022; A61B 5/14514; A61B 5/14532; A61B 5/1118; A61B 5/4806; A61B 5/021

(57) ABSTRACT

Wearable blood pressure biosensors, systems, and methods for short-term blood pressure predictions are disclosed herein. In some embodiments, a blood pressure prediction system is configured to provide short-term predictions of average blood pressures for future time period(s) or horizon(s) (e.g., for a coming week). The system can include models trained to predict future systolic values, diastolic blood pressure values, and/or trends. The models can be trained on data related to personalized body, health, and/or physical characteristics of the user, e.g., the current or previous blood pressure, amount of sleep, heart rate, blood glucose (BG), activity, weight, etc. In some embodiments, the models can also determine whether the blood pressure of the user will change or remain relatively constant over a period/range of time.

### CROSS-REFERENCE TO RELATED APPLICATION

The present application claims the benefit of U.S. Provisional Patent Application No. 63/188,641, filed April May 14, 2021, entitled SHORT-TERM BLOOD PRESSURE PREDICTION SYSTEMS AND METHODS, which is incorporated by reference herein in its entirety.

### TECHNICAL FIELD

This disclosure relates generally to personalized healthcare and, in particular, to wearable blood pressure biosensors, systems, and methods for short-term blood pressure predictions.

### BACKGROUND

Many individuals suffer from significant variations in blood pressure (BP) through short time spans. For example, systolic blood pressure and diastolic blood pressure can vary considerably throughout a 24-hour period for people with or with no known hypertension. Systolic and diastolic blood pressure can also reach levels as far as 10-20% off the daily average for most people. For most individuals, such fluctuations to blood pressure tend to occur simultaneously in systolic and diastolic levels due to their correlation. The average blood pressure along with the increased variability thereof is an important risk factor that can indicate changes in the normal functionality of the body of an individual. One such important indication is the possible development of heart diseases.

Both long-term and short-term blood pressure levels are thus of importance to the health of individuals. Tracking them may be beneficial to get early warnings on possible risk factors and undesirable changes to the body. Today, with the ease of measuring blood pressure at home via a designated cuff, or periodically with a phone or watch, the collection of short-term blood pressure averages allows for constant monitoring on the health of a user. However, conventional blood pressure monitoring systems can only provide the current and recent averages on blood pressure to give people a sense of how they are doing in the present. Thus, there is a need for improved systems and methods for determining what the blood pressure is likely to do in the future or coming period and give people an opportunity to take actions to prevent expected higher blood pressure in the future.

### BRIEF DESCRIPTION OF THE DRAWINGS

FIG. 1 is a schematic diagram of an exemplary computing environment in which a blood pressure prediction system operates, in accordance with embodiments of the present technology.

FIG. 2 is a schematic illustration of a biosensor device configured in accordance with embodiments of the present technology.

FIG. 3A is a flowchart illustrating a process for short-term blood pressure predictions, in accordance with embodiments of the present technology.

FIG. 3B is a flowchart illustrating a process for predicting blood pressure levels, in accordance with embodiments of the present technology.

FIG. 4A-4B illustrate examples of prompts and reinforcements output by the blood pressure prediction system configured in accordance with embodiments of the present technology.

FIG. 5 is a schematic block diagram of a computing system or device configure in accordance with embodiments of the present technology.

### DETAILED DESCRIPTION

The present technology generally relates to systems and methods for predicting blood pressure of individuals. In some embodiments, a blood pressure prediction system is configured to provide short-term predictions of average blood pressures for future time period(s) or horizon(s) (e.g., for a coming week). The system can include models trained to predict future systolic values, diastolic blood pressure values, and/or trends. The models can be trained on data related to personalized body, health, and/or physical characteristics of the user, e.g., the current or previous blood pressure, amount of sleep, heart rate, blood glucose (BG), activity, weight, etc. In some embodiments, the models can also determine whether the blood pressure of the user will change or remain relatively constant over a period/range of time. Body and/or health data can be used to estimate blood pressures for users ahead of time, and the system can lead users to a lifestyle that can help them reduce average blood pressure, keep their average blood pressures within desirable ranges, keep maximum blood pressure above or below a set value, etc. Accordingly, the system can use predicted values to guide individuals toward behavioral interventions that are likely to improve their health outcomes.

The systems disclosed herein can be used to identify datasets, determine relationships between datasets and blood pressure, determine weighting (e.g., weighting of data within a data set, weighting of individual data sets, etc.), and/or determine blood pressure related data. In some embodiments, the system can identify datasets, subsets of data, etc. For example, the systems can analyze a set of data and blood pressure data to identify a subset of the data and a relationship to blood pressure. Based on the relationship, the system can predict blood pressure based on a subset of measured data. For example, a first subset of data can be used to predict blood pressure during a first period (e.g., when awake, exercising, etc.) and a second subset of data can be used to predict blood pressure during a second period (e.g., when sleeping, resting, etc.). The number of types of data or datasets, weighting of data (or data sets, relationships between data (or sets of data), or the like) can be identified or determined based on one or more criteria. The criteria can be determined by the system, inputted by the user, or the like. The system can rank groups or buckets based on their contribution to blood pressure. Contributing factors can include the one or more contributing health factors include one or more of blood pressure, blood glucose, heart rate, food, activity, sleep, weight, medication, diagnosis, or demographics. The system can provide recommendations based on the rankings. For example, the system can develop an exercise schedule, exercise routines, sleeping schedule, or the like based on one or more goals. The system can identify events (e.g., exercise at optimal times of day, optimal length of exercise period, etc.) that contribute to achieving desired blood pressures or other goals.

As an example, the system can measure and identify a subset of data including the current/previous blood pressure values (e.g., maximal or minimal blood pressure values over the last month, 2 weeks), consumption/dietary routine (e.g., consumed carbs, cholesterol, alcohol, sodium in the past 5 days), sleep data (e.g., average hours of sleep, REM sleep data the past 7 days), and/or heart rate (e.g., maximal heart rate, average heart rate the past month) of a user. The system can predict, based on the subset of data, that the average systolic blood pressure of the user will increase the subsequent week (e.g., to 125 mmHg) and/or exceed an upper bound of a normal range for systolic blood pressure (e.g., exceeds 120 mmHg). In response, the system can recommend (e.g., send a notification to a user interface) self-care to the user, such as a number of exercise routines that can help lower the blood pressure of the user by a certain amount (e.g., 10 mmHg when the exercise is performed for a week) and prevent the increase predicted for the subsequent week. Examples of recommended exercise routines can include hiking, running, walking, treadmilling, bicycling, jump roping, skiing, skating, rowing, weight training, swimming, stretching, etc. The system can recommend the exercise be performed at certain times in a day/week/month (e.g., walking on Mondays, bicycling on weekends) and for certain lengths of time (e.g., 30 minutes a day of walking at night, 1 hour of stretching in the morning). In some embodiments, the system can recommend higher intensity activities (e.g., weight training, running, etc.) when appropriate given the predicted change in the blood pressure of the user. When the predicted increase in blood pressure falls into ranges that put the user at possible risk of hypertension, the system can send a notification to the user of the risk and identify lifestyle objectives to prevent or mitigate the risk (e.g., exercise routines to perform, dietary/consumption routines), or direct the user to seek a doctor or medical professional.

As another example, the system can also provide recommended consumption or dietary routines, physical activity or exercise routines, sleep behaviors/habits, heart rate or blood pressure changing activities, and/or insulin intake or dosages based on blood pressure objectives indicated by the user. The system can receive a goal set by the user that he/she would like to decrease or increase their blood pressure by a certain desired amount (e.g., 10 mmHg, 5 mmHg) over a future time period (e.g., the next day(s), week(s), month(s)). In response, the system can identify the optimal subset of features (e.g., the subset that achieves the highest rank or accuracy) that can achieve the desired amount of decrease/increase in blood pressure of the user. For example, the system can determine that a combination of increased hours of sleep (e.g., 2 more hours a day), earlier bed time (e.g., going to bed 30 minutes earlier), increased cardio or aerobic exercise (e.g., running or lifting weights), decreased dosages/intakes of insulin (80% of current dosage), a more balanced diet (e.g., fewer carbs, lowered LDL cholesterol, more protein), and/or drinking less alcohol (e.g., 1 less beer a day) can decrease the systolic or diastolic blood pressure in the coming week/month. The system can recommend self-care such as lifestyle changes, routines, and habits to the user to help him/her reach the goals and objectives. In some embodiments, the system can also check-up on whether the user has been following the recommendations. If the predicted blood pressure for the coming week has declined, for example, the system can determine that the user has been following the recommendations. In such instances, the system can recommend new recommendations or modify the previous recommendations to match the changes and improvements or to newly set objectives by the user. For example, the system may select a different subset of features (e.g., add or remove currently selected features) to recommend to the user or modify the current subset of features to recommend different amounts (e.g., 1 more hour of sleep a day, going to bed 15 minutes later, doing less intensive of an exercise).

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The headings provided herein are for convenience only and do not interpret the scope or meaning of the claimed present technology.

### Systems for Short-Term Blood Pressure Predictions

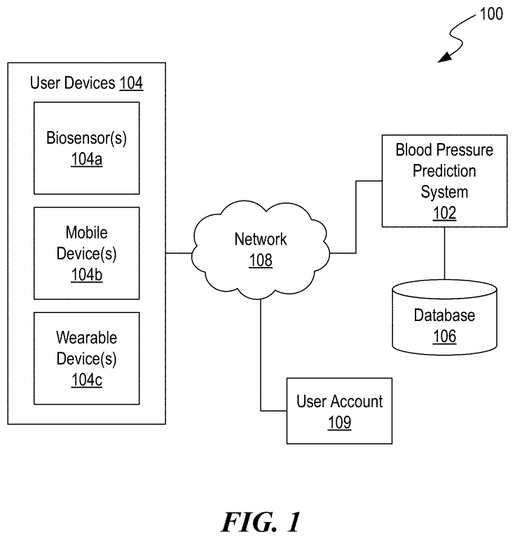

FIG. 1 is a schematic diagram of an exemplary computing environment 100 in which a blood pressure prediction system 102 (“system 102”) operates, in accordance with embodiments of the present technology. As shown in FIG. 1, the system 102 is operably coupled to one or more user devices 104 via a network 108. The system 102 is also operably coupled to at least one database or storage component 106 (“database 106”). The system 102 can include processors, memory, and/or other software and/or hardware components configured to implement the various methods described herein. For example, the system 102 can be configured to predict the future blood pressure averaged over a fixed/selected time range, as described in greater detail below.

The future blood pressure can be a systolic or diastolic blood pressure that is associated with the user. Systolic blood pressure can be the force the heart exerts on the walls of the arteries each time the heart beats, while diastolic blood pressure can be the force the heart exerts on the walls of the arteries in between heart beats. Both systolic and diastolic blood pressure can be measured in mmHg (millimeter of mercury). In some embodiments, the system 102 can forecast the average systolic or diastolic blood pressure over a time range of one week in advance. The techniques and procedures can also be used to predict the average blood pressure over other time periods/ranges or with other forecast horizons, such as one or more hours, days, months, or years in advance. In some embodiments, the system predicts a difference between the average systolic blood pressure and average diastolic blood pressure.

The input data for the system 102 can include health-related information, contextual information, and/or any other information relevant to the patient's health state. For example, health-related information can include levels or concentrations of a biomarker, such as glucose, blood glucose, electrolytes, neurotransmitters, amino acids, hormones, alcohols, gases (e.g., oxygen, carbon dioxide, etc.), creatinine, blood urea nitrogen (BUN), lactic acid, drugs, pH, cell count, and/or other biomarkers. Health-related information can also include physiological and/or behavioral parameters, such as vitals (e.g., heart rate, body temperature (such as skin temperature), blood pressure (such as systolic and/or diastolic blood pressure), respiratory rate), cardiovascular data (e.g., pacemaker data, arrhythmia data), body function data, meal/food or nutrition data (e.g., number of meals; timing of meals; number of calories; dietary information; amount of carbohydrates (carbs), fats, sugars, etc.), physical activity or exercise data (e.g., time and/or duration of activity; amount of calories burned from activity at every time period; activity type such as walking, running, swimming; strenuousness of the activity such as low, moderate, high; etc.), sleep data (e.g., number of hours of sleep, average hours of sleep, variability of hours of sleep, sleep-wake cycle data, data related to sleep apnea events, sleep fragmentation (such as fraction of nighttime hours awake between sleep episodes), bed time, etc.), stress level data (e.g., cortisol and/or other chemical indicators of stress levels, perspiration), a1c data, etc. Health-related information can also include medical history data (e.g., weight, height, waist circumference, age, sleeping patterns, medical conditions, cholesterol levels, disease type, family history, patient health history, diagnoses, tobacco usage, alcohol usage, static information about the user, etc.), diagnostic data (e.g., molecular diagnostics, imaging), medication data (e.g., timing and/or dosages of medications such as insulin), personal data (e.g., name, gender, demographics, social network information, etc.), basal energy expenditure, and/or any other data, and/or any combination thereof. Contextual information can include user location (e.g., GPS coordinates, elevation data), environmental conditions (e.g., air pressure, humidity, temperature, air quality, etc.), and/or combinations thereof.

The input data can be used to generate predictions, recommendations, guidance, and/or other information for assisting a user in monitoring and/or managing a disease, condition, or other health state, such as any of the following: hypertension, cardiovascular diseases, cardiovascular health, stress, trauma, drug use, physical performance, nutrition, mental and behavioral health, wellness, and/or combinations thereof. For example, the methods herein can be used to predict hypertension-related events, hypertension progression, hypotension-related events, and hypotension progression and then generate personalized guidance for actions that the user may take to improve, mitigate, and/or slow the progression of the disease or condition. As another example, the methods herein can be used to predict a user's future health state, and generate personalized guidance for actions to maintain and/or improve their health state. In yet another example, the methods herein can be used to predict whether a user will meet certain health goals, and generate personalized guidance for actions to increase the likelihood of meeting those health goals.

The system 102 can determine one or more blood pressure event predictions during a period of time. The blood pressure event predictions can be, for example, whether the blood pressure of the user will be one or more of the following: outside of an acceptable blood pressure range (e.g., a target systolic blood pressure range, a target diastolic blood pressure range, a user defined blood pressure range, etc.), below a threshold hypotension value, above a threshold hypertension value, or the like. In some embodiments, the blood pressure event prediction can be based on an average blood pressure over a period of time within a target range. If the average blood pressure is below or above a target blood pressure value, the system 102 can identify a blood pressure event prediction. The system 102 can generate a confidence score of the blood pressure event prediction based on confidence score criteria. In some embodiments, the blood pressure event prediction is a target blood pressure value associated with implementation of one or more contributing health factors. For example, the system 102 can generate one or more blood pressure event predictions based on whether the user completes recommended exercise activities. This allows the system 102 to provide the target blood pressure values and the recommended exercise activities.

The target blood pressure values can include, without limitation, one or more of target systolic blood pressure values, target diastolic pressure blood pressure values, target blood pressure values within a predetermined range, an average blood pressure value (e.g., an average diastolic blood pressure value, an average systolic blood pressure value, or combinations thereof), etc. The target blood pressure values can be selected based on tracking of the blood pressure of the user. The target blood pressure values can be selected based on, for example, one or more blood pressure goals. A blood pressure goals can include, for example, blood pressures within an acceptable range, blood pressures above/blow an predetermined value, etc.

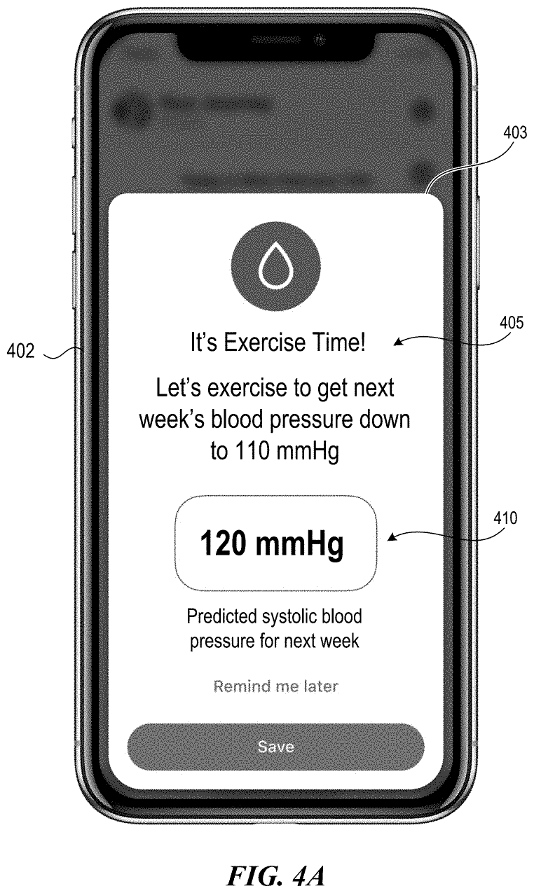

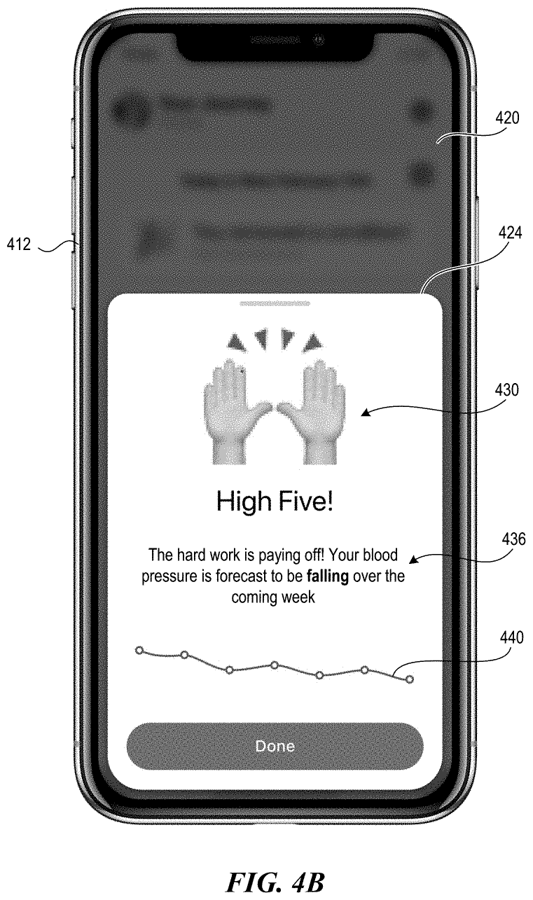

Referring now to FIG. 4A, the user device 402 can include, without limitation, an interface 403 displaying a notification with a recommendation 405. The recommendation 405 can include, without limitation, a prompter motivator indicating that it's time to exercise. The recommendation 405 can also include contributing health factors (e.g., sleep, diet, etc.) associated with a predicted reduction in blood pressure. In the illustrated embodiment, the notification 403 states that the user's blood pressure can be reduced to, for example, a predicted blood pressure or outcome 410 (e.g., 120 mm HG predicted systolic blood pressure for the next week in FIG. 4A). The user can determine whether to implement the recommendation 405 based on the predicted outcome 410. Advantageously, the system can generate the notification, recommend notification 403, recommendation 405, and prediction 410 using different data sources. This allows a user to monitor health data that may be affected by numerous health contributors.

Referring to FIG. 4B, the user device 412 includes an interface 420 that can display a GUI 424 with information related to blood pressure forecast. The GUI 424 can include prompts and/or reinforcements for promoting implementation of recommendations. The GUI 424 can include, for example, a prompt 430 indicating that implementation of one or more recommendations (e.g., dietary changes, exercise, sleep schedule, calm, etc.) for achieving one or more target outcomes, such a cardiovascular outcome, blood pressure outcome, weight loss outcome, etc. For example, the interface 420 can display information 436 and one or more forecasts of blood pressure values, (e.g., an average blood pressure, a maximum systolic/diastolic blood pressure, minimum blood pressure, etc.) over a period of time, such as, for example, the coming day(s), week(s), or month(s). The period of time can be the period in which a confidence score for forecasts (or predictions) are at or above a threshold confidence score value or another period of time selected by, for example, the user.

FIG. 4B shows an example forecast 440 in which the user's blood pressure is forecasted to be decreasing over the coming day(s)/week(s). Forecast 440 can include graphs showing the, for example, daily blood pressure averages, daily maximum/minimum blood pressure values, or combinations thereof. In some embodiments, the forecast 440 can indicate, for example, correlations between contributed health factors, recommendations, forecast, etc. In some embodiments, the forecast 440 can include multiple predictions, graphs, etc. for different scenarios (e.g., different exercise programs, dietary programs, etc.). The user can decide to implement one of the scenarios to achieve a desired outcome.

In some embodiments, the system 102 receives the input data from one or more user devices 104. The user devices 104 can be any device associated with a patient or other user, and can be used to obtain healthcare information, contextual information, and/or any other relevant information relating to the patient and/or any other users or patients (e.g., appropriately anonymized patient data). In the illustrated embodiment, for example, the user devices 104 include at least one biosensor 104a (e.g., blood pressure sensors, blood pressure measuring cuffs, blood glucose sensors, pressure sensors, heart rate sensors, sleep trackers, temperature sensors, motion sensors, or other biomonitoring devices), at least one mobile device 104b (e.g., a smartphone or tablet computer), and, optionally, at least one wearable device 104c (e.g., a smartwatch, fitness tracker, wearable blood pressure monitor, etc.). In other embodiments, however, one or more of the devices 104a-c can be omitted and/or other types of user devices can be included, such as computing devices (e.g., personal computers, laptop computers, etc.). Additionally, although FIG. 1 illustrates the biosensor(s) 104a as being separate from the other user devices 104, in other embodiments the biosensor(s) 104a can be incorporated into another user device 104.

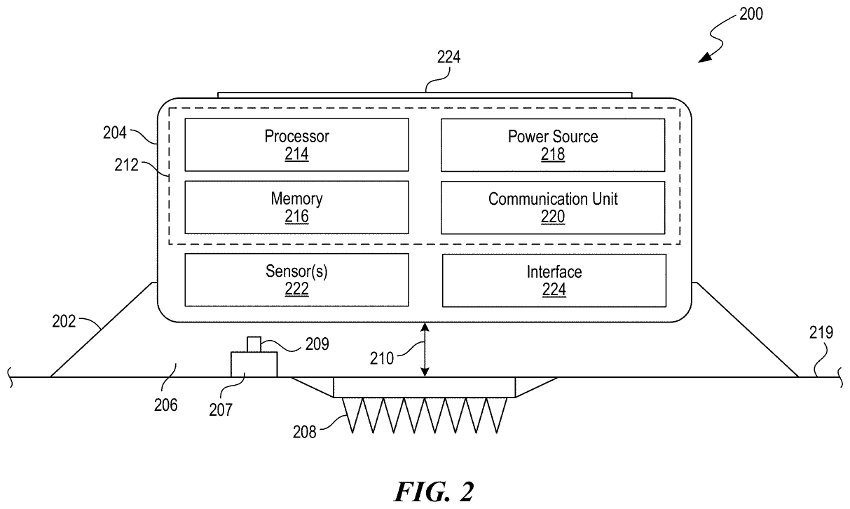

The biosensor 104a can include various types of sensors, such as chemical sensors, electrochemical sensors, optical sensors (e.g., optical enzymatic sensors, opto-chemical sensors, fluorescence-based sensors, etc.), spectrophotometric sensors, spectroscopic sensors, polarimetric sensors, calorimetric sensors, iontophoretic sensors, radiometric sensors, and the like, and combinations thereof. In some embodiments, the biosensor 104a is or includes a blood pressure sensor. The blood pressure sensor can be any device capable of obtaining blood pressure data from the patient, such as non-invasive sensors, minimally invasive sensors, invasive sensors, implanted sensors, non-implanted sensors, wearable sensors, etc. The blood pressure sensor can be configured to obtain values of blood pressure with a palpation method; (e.g., via sphygmomanometer and palpating the radial pulse); auscultatory method (e.g., using a stethoscope and sphygmomanometer, inflatable cuff placed around arm, attached to a mercury or aneroid manometer); oscillometric method (e.g., using a sphygmomanometer cuff with an electronic pressure sensor (transducer) to observe pressure oscillations); continuous noninvasive aerial pressure technique (e.g., measures blood pressure continuously like invasive arterial catheter, but is noninvasive); pulse wave velocity technique (e.g., translating pulse wave velocity values into blood pressure values); ambulatory and home monitoring (e.g., taking readings regularly throughout a fixed/selected period, automatic self-contained blood pressure monitors); and/or usage of arterial lines (e.g., placing cannula needle in artery via palpation). For example, the blood pressure sensor can include one or more pulsoxymeters, photoplethysmography (PPG) sensors, actuatable sensors, combinations thereof, or the like. Optionally, the blood glucose sensor can include other capabilities, such as processing, transmitting, receiving, and/or other computing capabilities. Example biosensors with blood pressure sensors and blood glucose sensor are discussed in connection with FIG. 2.

In some embodiments, some or all of the user devices 104 are configured to continuously obtain any of the above data (e.g., health-related information and/or contextual information) from the patient over a particular time period (e.g., hours, days, weeks, months, years). For example, data can be obtained at a predetermined time interval (e.g., once every minute, 2 minutes, 5 minutes, 10 minutes, 15 minutes, 20 minutes, 30 minutes, 60 minutes, 2 hours, etc.), at random time intervals, user-set intervals, regular intervals, irregular intervals, or combinations thereof. The time interval for data collection can be set by the patient, by another user (e.g., a physician), by the system 102, or by the user device 104 itself (e.g., as part of an automated data collection program). The user device 104 can obtain the data automatically or semi-automatically (e.g., by automatically prompting the patient to provide such data at a particular time), or from manual input by the patient (e.g., without prompts from the user device 104). The continuous data may be provided to the system 102 at predetermined time intervals (e.g., once every minute, 2 minutes, 5 minutes, 10 minutes, 15 minutes, 20 minutes, 30 minutes, 60 minutes, 2 hours, etc.), continuously, in real-time, upon receiving a query, manually, automatically (e.g., upon detection of new data), semi-automatically, etc. The time interval at which the user device 104 obtains data may or may not be the same as the time interval at which the user device 104 transmit the data to the system 102.

The user devices 104 can obtain any of the above data and can provide output in various ways, such as using one or more of the following components: a microphone (either a separate microphone or a microphone imbedded in the device), a speaker, an interface, a screen (e.g., using a touchscreen, a stylus pen, and/or in any other fashion), a keyboard, a mouse, a camera, a camcorder, a telephone, a smartphone, a tablet computer, a personal computer, a laptop computer, a sensor (e.g., a sensor included in or operably coupled to the user device 104), and/or any other device. The data obtained by the user devices 104 can include metadata, structured content data, unstructured content data, embedded data, nested data, hard disk data, memory card data, cellular telephone memory data, smartphone memory data, main memory images and/or data, forensic containers, zip files, files, memory images, and/or any other data/information. The data can be in various formats, such as text, numerical, alpha-numerical, hierarchically arranged data, table data, email messages, text files, video, audio, graphics, etc. Optionally, any of the above data can be filtered, smoothed, augmented, annotated, or otherwise processed (e.g., by the user devices 104 and/or the system 102) before being used.

In some embodiments, any of the above data can be queried by one or more of the user devices 104 from one or more databases (e.g., the database 106, a third-party database, etc.). The user device 104 can generate a query and transmit the query to the system 102, which can determine which database may contain requisite information and then connect with that database to execute a query and retrieve appropriate information. In other embodiments, the user device 104 can receive the data directly from the third-party database and transmit the received data to the system 102, or can instruct the third-party database to transmit the data to the system 102. In some embodiments, the system 102 can include various application programming interfaces (APIs) and/or communication interfaces that can allow interfacing between user devices 104, databases, and/or any other components.

Optionally, the system 102 can also obtain any of the above data from various third party sources, e.g., with or without a query initiated by a user device 104. In some embodiments, the system 102 can be communicatively coupled to various public and/or private databases that can store various information, such as census information, health statistics (e.g., appropriately anonymized), demographic information, population information, and/or any other information. Additionally, the system 102 can also execute a query or other command to obtain data from the user devices 104 and/or access data stored in the database 106. The data can include data related to the particular patient and/or a plurality of patients or other users (e.g., health-related information, contextual information, etc.) as described herein.

The database 106 can be used to store various types of data obtained and/or used by the system 102. For example, any of the above data can be stored in the database 106. The database 106 can also be used to store data generated by the system 102, such as previous predictions or forecasts produced by the system 102. In some embodiments, the database 106 includes data for multiple users, such as a plurality of patients (e.g., at least 50, 100, 200, 500, 1000, 2000, 3000, 4000, 5000, or 10,000 different patients). The data can be appropriately anonymized to ensure compliance with various privacy standards. The database 106 can store information in various formats, such as table format, column-row format, key-value format, etc. (e.g., each key can be indicative of various attributes associated with the user and each corresponding value can be indicative of the attribute's value (e.g., measurement, time, etc.)). In some embodiments, the database 106 can store a plurality of tables that can be accessed through queries generated by the system 102 and/or the user devices 104. The tables can store different types of information (e.g., one table can store blood pressure measurement data, another table can store user health data, etc.), where one table can be updated as a result of an update to another table. The database 106 can also include, without limitation, one or more training data sets (e.g., machine-learning training data sets), verification data sets, and other data sets.

In some embodiments, one or more users can access the system 102 via the user devices 104, e.g., to send data to the system 102 (e.g., health-related information, contextual information) and/or receive data from the system 102 (e.g., predictions, notifications, recommendations, instructions, support, etc.). The users can be individual users (e.g., patients, healthcare professionals, etc.), computing devices, software applications, objects, functions, and/or any other types of users and/or any combination thereof. For example, upon obtaining any of the input data discussed above, the user device 104 can generate an instruction and/or command to the system 102, e.g., to process the obtained data, store the data in the database 106, extract additional data from one or more databases, and/or perform analysis of the data. The instruction/command can be in a form of a query, a function call, and/or any other type of instruction/command. In some implementations, the instructions/commands can be provided using a microphone (either a separate microphone or a microphone imbedded in the user device 104), a speaker, a screen (e.g., using a touchscreen, a stylus pen, and/or in any other fashion), a keyboard, a mouse, a camera, a camcorder, a telephone, a smartphone, a tablet computer, a personal computer, a laptop computer, and/or using any other device. The user device 104 can also instruct the system 102 to perform an analysis of data stored in the database 106 and/or inputted via the user device 104.

As discussed further below, the system 102 can analyze the obtained input data, including historical data, current real-time data, continuously supplied data, and/or any other data (e.g., using a statistical analysis, machine learning analysis, etc.), and generate output data. The output data can include the systolic or diastolic blood pressure averaged over a fixed/selected time range, or an indication of whether the blood pressure of the user will rise, fall, or remain steady over a future time period/range (e.g., class label). In some embodiments, the output data can also include predictions of a patient's health state, interpretations, recommendations, notifications, instructions, support, and/or other information related to the obtained input data. The system 102 can perform such analyses at any suitable frequency and/or any suitable number of times (e.g., once, multiple times, on a continuous basis, etc.). For example, when updated input data is supplied to the system 102 (e.g., from the user devices 104), the system 102 can reassess and update its previous output data, if appropriate. In performing its analysis, the system 102 can also generate additional queries to obtain further information (e.g., from the user devices 104, the database 106, or third party sources). In some embodiments, the user device 104 can automatically supply the system 102 with such information. Receipt of updated/additional information can automatically trigger the system 102 to execute a process for reanalyzing, reassessing, or otherwise updating previous output data.

In some embodiments, the system 102 is configured to analyze the input data and generate the output data using one or more machine learning models. The machine learning models can include supervised learning models, unsupervised learning models, semi-supervised learning models, and/or reinforcement learning models. Examples of machine learning models suitable for use with the present technology include, but are not limited to: regression algorithms (e.g., ordinary least squares regression, linear regression, logistic regression, stepwise regression, multivariate adaptive regression splines, locally estimated scatterplot smoothing), instance-based algorithms (e.g., k-nearest neighbor, learning vector quantization, self-organizing map, locally weighted learning, support vector machines), regularization algorithms (e.g., ridge regression, least absolute shrinkage and selection operator, elastic net, least-angle regression), decision tree algorithms (e.g., classification and regression trees, Iterative Dichotomiser 3 (ID3), C4.5, C5.0, chi-squared automatic interaction detection, decision stump, M5, conditional decision trees), Bayesian algorithms (e.g., naïve Bayes, Gaussian naïve Bayes, multinomial naïve Bayes, averaged one-dependence estimators, Bayesian belief networks, Bayesian networks), clustering algorithms (e.g., k-means, k-medians, expectation maximization, hierarchical clustering), association rule learning algorithms (e.g., apriori algorithm, ECLAT algorithm), artificial neural networks (e.g., perceptron, multilayer perceptrons, back-propagation, stochastic gradient descent, Hopfield networks, radial basis function networks), deep learning algorithms (e.g., convolutional neural networks, recurrent neural networks, long short-term memory networks, stacked auto-encoders, deep Boltzmann machines, deep belief networks), dimensionality reduction algorithms (e.g., principle component analysis, principle component regression, partial least squares regression, Sammon mapping, multidimensional scaling, projection pursuit, discriminant analysis), time series forecasting algorithms (e.g., exponential smoothing, autoregressive models, autoregressive with exogenous input (ARX) models, autoregressive moving average (ARMA) models, autoregressive moving average with exogenous inputs (ARMAX) models, autoregressive integrated moving average (ARIMA) models, autoregressive conditional heteroskedasticity (ARCH) models), and ensemble algorithms (e.g., boosting, bootstrapped aggregation, AdaBoost, blending, stacking, gradient boosting machines, gradient boosted trees, random forest).

The computing environment 100 can further include a user account 109 that can be linked with user devices 104, the blood pressure prediction system 102, or other devices or systems disclosed herein. A user can use a web portal, mobile app, or another means to link to data sources or accounts (e.g., health tracker account, dietary logging system account, exercise tracker account, etc.). In some embodiments, the user can authorize and allow access and/or retrieval of additional health data from these linked data sources or accounts. In some embodiments, the user can authorize the user account 109 (e.g., a cloud based account) to access and retrieve health data from one or more of user devices, such as the wearable devices 104c, biosensors 104a, and other devices 104 of FIG. 1, and/or device 200 of FIG. 2. The user account 109 can periodically or continuously retrieve data and transmit all or a portion of the data to, for example, trained machine learning models, input data to machine learning models, generate values (e.g., forecast, predictions, etc.) disclosed herein.

The blood pressure prediction system 102 can communicate, via the network 108, to retrieve the available healthcare data. The blood prediction system 102 can also send settings, request, data acquisition information to the user devices 104 to control the health data being collected. This allows the system 100 to adjust the health data collection based on the monitoring to be performed. The blood pressure prediction system 102 can receive health data or output from the user account 109. In some embodiments, the blood pressure prediction system 102 can retrieve datasets with a data type that matches a data type of training sets used to train machine learning models. For example, the blood pressure prediction system 102 can retrieve glucose level data from a continuous glucose monitor based on the machine learning models being trained using continuous glucose monitoring data training sets. This allows a blood pressure prediction system 102 to analyze available user devices 104, identify available data types that match data types used for training, and retrieval of all or a portion of the available user data to input into a machine learning model that was trained using similar data. This allows the blood pressure prediction system 102 to selectively acquire health data identified to be suitable for input into the machine learning models. Example types of available health data can include, without limitation, blood pressure data, sleep data, blood glucose data, user activity data, dietary data, or the like. In some embodiments, the user device is 104 communicator sync with one another to share available health data.

Although FIG. 1 illustrates a single set of user devices 104, it will be appreciated that the system 102 can be operably and communicably coupled to multiple sets of user devices, each set being associated with a particular patient or user. Accordingly, the system 102 can be configured to receive and analyze data from a large number of patients (e.g., at least 50, 100, 200, 500, 1000, 2000, 3000, 4000, 5000, or 10,000 different patients) over an extended time period (e.g., weeks, months, years). The data from these patients can be used to train and/or refine one or more machine learning models implemented by the system 102, as described below.

The system 102 and user devices 104 can be operably and communicatively coupled to each other via the network 108. The network 108 can be or include one or more communications networks, and can include at least one of the following: a wired network, a wireless network, a metropolitan area network (“MAN”), a local area network (“LAN”), a wide area network (“WAN”), a virtual local area network (“VLAN”), an internet, an extranet, an intranet, and/or any other type of network and/or any combination thereof. Additionally, although FIG. 1 illustrates the system 102 as being directly connected to the database 106 without the network 108, in other embodiments the system 102 can be indirectly connected to the database 106 via the network 108. Moreover, in other embodiments one or more of the user devices 104 can be configured to communicate directly with the system 102 and/or database 106, rather than communicating with these components via the network 108.

The various components 102-108 illustrated in FIG. 1 can include any suitable combination of hardware and/or software. In some embodiment, components 102-108 can be disposed on one or more computing devices, such as, server(s), database(s), personal computer(s), laptop(s), cellular telephone(s), smartphone(s), tablet computer(s), and/or any other computing devices and/or any combination thereof. In some embodiments, the components 102-108 can be disposed on a single computing device and/or can be part of a single communications network. Alternatively, the components can be located on distinct and separate computing devices. For example, although FIG. 1 illustrates the system 102 as being a single component, in other embodiments the system 102 can be implemented across a plurality of different hardware components at different locations.

### Wearable Blood Pressure Biosensors

FIG. 2 is a schematic illustration of a biosensor device 200 (“device 200”) configured in accordance with embodiments of the present technology. The device 200 can be configured to be applied to a user's body in order to obtain user health data in a non-invasive manner and/or minimally-invasive manner. The device 200 can be used in any of the systems and methods described herein (e.g., as the biosensor 104a of FIG. 1). The device 200 can generate health data of a user that is transmitted to a computing device that sends information to be provided to the user via, for example, an interface 224. The interface 224 can be a screen that displays, for example, forecasts, predictions, notification, goals, and health-related information to the user.

The device 200 can include a patch 202 (also referred to as a “patch portion,” “base portion,” or “sensing component”) and a pod 204 (also referred to as a “pod portion,” “capsule portion,” or “electronics component”). The patch 202 can be coupled to the pod 204 (e.g., releasably coupled or permanently affixed) to form the device 200. The patch 202 can include a substrate 206 configured to couple to the user's body (e.g., to the surface of the skin) via adhesives or other suitable temporary attachment techniques. The base portion also includes a blood pressure sensor 207 configured to detect, for example, health data, including biometric parameters indicative of blood pressure, cardiovascular dynamics (e.g., continuous or periodic real-time cardiovascular dynamics), hemodynamic variables, or the like. In some embodiments, the blood pressure sensor 207 can detect real-time blood pressure values (e.g., maximal or minimal blood pressure values over a period of time, interview, etc.), or other values disclosed herein. The patch 202 can include an actuator 209 configured to move the sensor 207 away from or toward a user's skin to maintain contact, target pressure, etc. The actuator 209 can also function as a pressure sensor to measure contact pressure applied to the skin. The configuration, detection capability, and function of the blood pressure sensor 207 can be selected based on the desired blood pressure measurements.

The patch 202 can also include at least one array of microneedles 208 coupled to and/or supported by the substrate 206 and configured to generate health data. The microneedles 208 can be configured to penetrate into the user's skin 219 to access interstitial fluid therein. In some embodiments, when the device 200 is applied to the skin, the microneedles 208 extend only into the stratum corneum and epidermis, and do not penetrate into the dermis or hypodermis (subcutaneous tissue). This approach can reduce or avoid pain and/or discomfort, while still providing accurate detection of analytes in the epidermal interstitial fluid. The microneedles 208 can be analyte-detecting microneedles configured to detect one or more analytes in the interstitial fluid when the microneedles extend into the user's skin. The analytes can include, without limitation, glucose, gases, electrolytes, BUN, creatinine, ketones, alcohols, amino acids, neurotransmitters, hormones, biomarkers, drugs, pH, cell count, and/or any of the other analytes described herein. Each microneedle 208 can be configured to detect a single analyte, or some or all of the microneedles 208 can be configured to detect multiple analytes (e.g., two, three, four, five, or more different analytes). Optionally, some or all of the microneedles 208 can be configured to detect physiological parameters, such as electrical properties (e.g., biopotential, bioimpedance), body temperature, etc.

The array can include any suitable number of microneedles 208 (e.g., 25 microneedles), and the microneedles 208 can be arranged in any suitable geometry (e.g., a 5×5 grid). Although FIG. 2 illustrates a single array of microneedles 208, in other embodiments, the device 200 can include two, three, four, five, or more arrays of microneedles 208. In embodiments where the device 200 includes multiple arrays, each array can be configured to perform a different function, or some of the arrays can perform the same function. For example, the device 200 can include a first array configured to detect a first set of analytes, a second array configured to detect a second set of analytes, a third array configured to detect a third set of analytes, and so on. Alternatively or in combination, the device 200 can include a first array configured as a working electrode, a second array configured as a reference electrode, and a third array configured as a counter electrode. Additional details and examples of microneedles and microneedle arrays suitable for use in the device 200 are described below in Section III.

The array of microneedles 208 can generate signals (e.g., electrical signals) indicative of health parameter values (e.g., analyte concentration and/or physiological values). For example, the array of microneedles 208 can generate a first electrical signal indicative of a first analyte, a second electrical signal indicative of a second analyte, and so on. Optionally, the array of microneedles 208 can generate at least a first electrical signal indicative of an analyte and at least a second electrical signal indicative of a physiological parameter. The array of microneedles 208 can be electrically coupled to the patch 202, which in turn can be electrically coupled to the pod 204 (schematically represented by arrow 210). The electrical connections between the array of microneedles 208, patch 202, and pod 204 can include any suitable combination of pins, contacts, wires, traces, etc. Accordingly, the signals generated by the microneedles 208 can be transmitted to the pod 204 for storage and/or processing.

The pod 204 can be a capsule, module, or other durable structure that couples to the patch 202 in order to assemble the device 200. The pod 204 can be mechanically coupled to the patch 202 using any suitable temporary or permanent attachment method, such as interference fit, snap fit, threading, fasteners, bonding, adhesives, and/or suitable combinations thereof. The pod 204 can include a casing or housing that encloses an electronics assembly 212 (also referred to herein as an “electronics subsystem”) of the device 200. The electronics assembly 212 can include one or more electronic components configured to perform the various operations described herein, such as a processor 214, memory 216, power source 218, and communication unit 220. Optionally, the pod 204 can also include one or more sensors 222 for measuring physiological parameters. The pod 204 can also include other electronic components not shown in FIG. 2, such as additional signal processing circuitry (e.g., multiplexer, analog front end (AFE), amplifier, filter, analog-to-digital converters (ADCs)), clock circuitry, power management circuitry, user input/output devices, and the like.

The processor 214 can be any component suitable for controlling the operations of the device 200, such as a microprocessor, microcontroller, field-programmable gate array (FPGA), application-specific integrated circuit (ASIC), and the like. For example, the processor 214 can receive and process signals generated by the array of microneedles 208 and/or the sensor(s) 222 in order to generate one or more measurements of health parameters (e.g., analyte levels, biopotential values, bioimpedance values, body temperature values, heart rate values, oxygen levels, etc.). In some embodiments, the processor 214 receives and processes electrical signals from the blood pressure sensor 207. The processor 214 can also receive and process at least a first electrical signal from the array of microneedles 208 to generate a first health measurement (e.g., an analyte level), and at least a second electrical signal from the sensor(s) 222 to generate a second health measurement (e.g., a physiological parameter). The processor 214 can be configured to receive and process any number of electrical signals (e.g., two, three, four, five, or more electrical signals) obtained by different sensing components of the device 200 to generate measurements of multiple health parameters (e.g., two, three, four, five or more different health parameters). Optionally, the processor 214 can use the health measurements to generate predictions, recommendations, notifications, etc. As another example, the processor 214 can control transmission of raw sensor data, processed data, health measurements, predictions, etc., to a remote device (e.g., a smartphone, smartwatch, or other user device or remote server). In a further example, the processor 214 can receive instructions from a remote device for controlling the operation of the device 200 (e.g., powering on, powering off, updating calibration and/or other signal processing parameters, device pairing, etc.). The processor 214 can also control the operations of the other components of the device 200 (e.g., operations of the memory 216, power source 218, communication unit 220, other sensor(s) 222, etc.).

The memory 216 can store instructions to be executed by the processor 214 and/or data generated during operation of the device 200. For example, the memory 216 can store raw and/or processed sensor data, as well as generated health measurements, predictions, recommendations, notifications, etc. The memory 216 can also store operating parameters for the device 200, such as calibration parameters, signal processing parameters, algorithms or programs (e.g., for generating health measurements, predictions, etc.), and so on. The memory 216 can include any suitable combination of volatile and non-volatile memory, such as flash memory, EEPROM, etc. In some embodiments, the memory 216 can store executable instructions to adjust operation of the biosensor device 200 based on, for example, one or more user inputs, forecasts, blood pressure event predictions, contributing health factors, combinations thereof, or the like. The instructions can be received via over-the-air updates, over-the-air downloads, or wired communications (e.g., when synced with a computing device, such as a smartphone, computer, tablet, etc.).

The memory 216 can store blood pressure monitoring settings. In some embodiments, the electronics assembly 212 and/or blood pressure prediction system (e.g., blood pressure prediction system 102 of FIG. 1) can determine blood pressure monitoring settings based on output from the at least one machine-learning model to achieve a threshold confidence score for additional predictions. The electronics assembly 212, user devices (e.g., user devices 104 of FIG. 1), and/or blood pressure prediction systems can evaluate one or more predictions based on the transmitted blood pressure monitoring settings used by the biosensor device.

The power source 218 can be any component suitable for powering the operations of the device 200, such as a rechargeable battery, non-rechargeable battery, or suitable combinations thereof. The power source 218 can output power to the array of microneedles 208, processor 214, memory 216, communication unit 220, sensor(s) 222, and/or any other electronic components on the patch 202 or pod 204. The power source 218 can include or be operably coupled to power management circuitry (not shown). The power management circuitry can detect the charge status of the power source 218 (e.g., fully charged, partially charged, low charge), can allow the device 200 to operate in various modes (e.g., low power, full power), and/or any other suitable power-related function.

The communication unit 220 can allow the device 200 to transmit data to and/or receive data from a remote device (e.g., a mobile device, smartwatch, remote server, etc.). The communication unit 220 can be configured to communicate via any suitable combination of wired and/or wireless communication modes. In some embodiments, for example, the communication unit 220 uses Bluetooth Low Energy (BLE) to transmit and receive data.

The sensor(s) 222 can include any suitable combination of sensors for monitoring various health parameters, such as an optical sensor (e.g., PPG sensor, pulse oximeter), heart rate sensor, blood pressure sensor, electrocardiogram (ECG) sensor, activity or motion sensor (e.g., accelerometer, gyroscope), temperature sensor (e.g., thermistor), location sensor, humidity sensor, etc. Each sensor can generate a respective set of signals, which can be received and processed by the processor 214 to generate health measurements and/or other user data. In some embodiments, the device 200 includes at least one, two, three, four, five, or more different sensors 222 for measuring physiological and/or other user parameters. Each sensor 222 can be located at any suitable region of the pod 204, such as at or near an upper surface, lower surface, lateral surface, or within an interior cavity of the pod 204. In other embodiments, however, some or all of the sensor(s) 222 can instead be located in the patch 202, rather than in the pod 204. For example, a temperature sensor can be located in the patch 202 in order to generate measurements of the user's skin temperature.

In some embodiments, the patch 202 is a disposable component that is configured for short-term use (e.g., no more than 4 weeks, 3 weeks, 2 weeks, 1 week, 6 days, 5 days, 4 days, 3 days, 2 days, 1 day, 12 hours, etc.), while the pod 204 is a reusable component that is configured for longer-term use (e.g., at least 1 week, 2 weeks, 3 weeks, 4 weeks, 1 month, 2 months, 3 months, 6 months, 1 year, etc.). This approach can be advantageous for reducing overall cost of the device 200, particularly in embodiments where the pod 204 includes more expensive components (e.g., the electronics assembly 212 and/or other sensor(s) 222). In such embodiments, the reusable pod 204 can be coupled to the disposable patch 202 to assemble the device 200 for use, and can be decoupled from the disposable patch 202 when the disposable patch 202 is to be replaced. As such, a single reusable pod 204 can be used with multiple different disposable patches 202, which can reduce the overall cost of the device 200, and enhance device longevity and adaptability. Optionally, a single reusable pod 204 can be used with multiple disposable patches 202 that detect different types of analytes. For example, the reusable pod 204 can be configured to interface with a first disposable patch 202 configured to detect a first set of analytes, a second disposable patch 202 configured to detect a second set of analytes, a third disposable patch 202 configured to detect a third set of analytes, and so on. In other embodiments, however, the patch 202 and pod 204 can both be disposable components, or can both be reusable components.

The device 200 can be configured to obtain and process the signals generated by the array of microneedles 208 and/or the sensor(s) 222 in order to determine measurements for one or more health parameters, such as measurements of glucose, gases, electrolytes, BUN, creatinine, ketones, cholesterol, alcohols, amino acids, neurotransmitters, hormones, disease biomarkers, drugs, pH, cell count, heart rate, body temperature, blood pressure, respiratory rate, cardiovascular data, body function data, meal or nutrition data, physical activity or exercise data, sleep data, stress level data, a1c data, and so on. In some embodiments, the electronics assembly 212 is configured to implement one or more algorithms, such as algorithms for sensor calibration, signal conditioning, determining presence of and/or values for health parameters based on the sensor signals, predicting current and/or future values for health parameters based on the sensor signals, etc. The algorithms can be stored locally at the electronics assembly 212 (e.g., in the memory 216) such that the device 200 can operate without being in communication with a separate computing device or system (e.g., a cloud computing network, remote server, user device, etc.). In such embodiments, the locally-stored algorithms can be periodically updated, e.g., via firmware updates and/or other modifications received from the separate computing device by the communication unit 220. Alternatively or in combination, some or all of the algorithms can be stored at the separate computing device or system. In some embodiments, local processing can be performed onboard the device 200 for certain situations (e.g., when network connectivity is lost), while processing can be performed at a separate computing device or system in other situations (e.g., when network connectivity is available).

The operation of the device 200 can be customized based on the particular health parameters to be detected. For example, the patch 202 can include a respective memory (not shown) configured to store identifier information for the patch 202, such as the type and/or configuration of the pressure sensor 207, microneedles 208, the type and/or configuration of the microneedle arrays, the types of analytes and/or physiological parameters detected by the microneedles 208, the types of other sensors included in the patch 202, a unique patch ID (e.g., a serial number), a lot ID, manufacturing date, expiration date and/or expected lifetime, and/or any other suitable information. In some embodiments, the processor 214 is configured to detect when the pod 204 is coupled to the patch 202. Once the pod 204 is connected to the patch 202, the processor 214 can interrogate or otherwise communicate with the patch 202 to detect the identifier information for the patch 202. The processor 214 can access and read the identifier information, and can then adjust the parameters and/or algorithms used to process the electrical signals generated by the patch 202 (e.g., by the microneedles 208), based on the identifier information. For example, the processor 214 can use the identifier information to determine detection capabilities of the patch 202 (e.g., which analytes and/or physiological values the patch 202 is configured to detect). The processor 214 can select an appropriate locally-stored algorithm for processing the signals generated by the patch 202 and/or determining health parameters from the signals. The algorithms can include one or more blood pressure-prediction algorithms for measuring blood-pressure related metrics, determining blood pressure (e.g., based on output from multiple sensors, sensor 207 alone, etc.), calibrating sensors, filtering sensor data, etc. The algorithm can vary depending on, for example, current state of the user, the sensor type and/or configuration, type of detected analyte or parameter, the manufacturing information for the patch 202 (e.g., batch or lot ID), the expected lifetime of the patch 202, other available sensor data, or any other suitable factor. Additionally, parameter detection can be performed using different algorithms used with different groups of users and algorithms selected based on user health data. The locally-stored algorithm s can be updated based on the health parameters (e.g., via updates received from a separate user device, cloud computing system, etc.).

In embodiments where the pod 204 is configured for use with multiple patches 202 having different functionalities (e.g., different detection capabilities), when the pod 204 is coupled to a new patch 202, the processor 214 can use the identifier information received from the patch 202 to assess the functionality of the patch 202. If the processor 214 determines that the patch 202 has newly available functionality that the processor 214 is not currently programmed to accommodate, the processor 214 can retrieve the appropriate algorithms, calibration parameters, signal processing parameters, and/or other updates from a remote device (e.g., a user device, cloud computing system, etc.). Accordingly, the software implemented by the pod 204 can be rapidly and dynamically updated to accommodate different and/or new patch functionalities.

The health measurements produced by the device 200 can be used to generate personalized healthcare tracking and guidance, such as one or more predictions, recommendations, suggestions, feedback, and/or diagnosis for a number of diseases, conditions, or health states. For example, blood pressure can be monitored and/or predicted based on optical data (e.g., PPG data), electrical data (e.g., ECG data), heart rate data, user data, and/or activity data. As another example, sleep (e.g., sleep patterns, sleep quality) can be tracked and/or predicted based on heart rate data, skin temperature data, and/or activity data. In a further example, respiratory illness (e.g., COVID-19, allergies, infections etc.) can be monitored and/or predicted based on skin temperature data, blood pressure data, and/or respiration rate. The health measurements can be used to detect a condition, distinguish between different conditions (e.g., infection versus allergies), and/or monitor the progression of the condition. In yet another example, fertility can be tracked and/or predicted based on skin temperature data. The personalized guidance can be generated based solely on the health measurements from the device 200, or can be generated through a combination of health measurements and other information (e.g., information from any number of sensor data streams, user data sets, etc.). The healthcare guidance can be generated locally onboard the device 200, by a user device that receives health measurement data from the device 200 (e.g., via a mobile application on a user's smartphone or smartwatch), by a cloud computing system or remote server that receives health measurement data from the device 200, or any suitable combination thereof.

The configuration of the device 200 shown in FIG. 2 can be modified in many different ways. For example, in other embodiments, the array of microneedles 208 can be omitted such that the device 200 does not include or otherwise use microneedle-based analyte detection. In such embodiments, the patch 202 may include other sensor types (e.g., a temperature sensor, a metal electrode for ECG sensing), or may not include any analyte sensors at all, such that all sensing operations are performed by the sensor(s) 222 (e.g., motion sensor, optical sensor, etc.) located in the pod 204. As another example, any of the components of the device 200 can be separated into discrete subcomponents (e.g., multiple processors 214, multiple memories 216, etc.), combined into a single component (e.g., the processor 214 and communication unit 220 can be integrated into a single chip), or omitted altogether. In a further example, any of the components of the device 200 can be positioned at different locations (e.g., some or all of the sensor(s) 222 can be located on the patch 202 instead of the pod 204).

The device 200 can include an interface 224 configured to provide output. The interface 224 can include, without limitation, an electronic display, a liquid crystal display, a touchscreen or another user input device configured to accept tactile input. In some embodiments, the interface 224 is coupled to the electronics assembly 212 and displays, for example, health data, notifications, predictions, health factors, recommendations, and other information. For example, the interface 224 can display the information discussed in connection with FIGS. 4A and 4B. The feedback provided to the user can be any form of sensory feedback, such as for example visual feedback, auditory feedback, or tactile feedback; and input from the user can be received in any form, including, but not limited to, acoustic, speech, or tactile input. In some embodiments, the device 200 provides non-visual output, such as audible notifications. The notifications can indicate adverse event prediction. The interface can visually display data associated with the adverse event prediction.

### Methods for Short-Term Blood Pressure Predictions

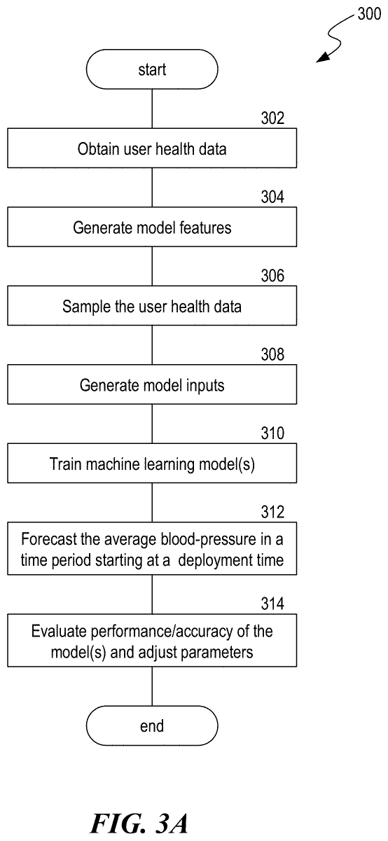

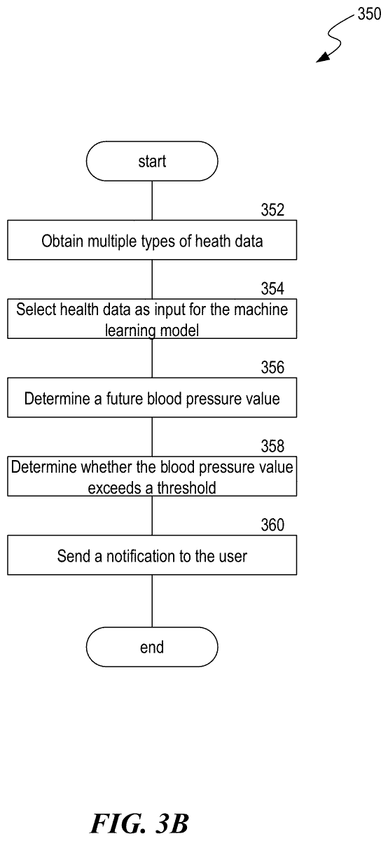

In some embodiments, the blood pressure prediction systems described herein (e.g., the system 102 of FIG. 1) and devices (e.g., the device 200 of FIG. 2) are configured to provide predictions of a blood pressure average (e.g., a daytime blood pressure average, a nighttime blood pressure average, a weekly blood pressure average, a blood pressure average for an activity, a user selected blood pressure average, etc.) for future time periods or horizons, predict changes in average blood pressure, and/or predict when the average blood pressure of the user will reach a predetermined value. In some embodiments, the system can detect possible future changes, increases, and/or decreases to the blood pressure of a user. FIG. 3A is a block diagram illustrating a process 300 for short-term blood pressure predictions, in accordance with embodiments of the present technology.

At step 302, the system can first obtain raw input data (user health data) involving a number of data types related to the body and health of users. The raw input data can include any of the input data described above in relation to system 102 and/or database 106 of FIG. 1. For example, the data types can include: blood pressure (e.g., systolic and/or diastolic blood pressure), sleep (e.g., number of hours of sleep, bed time, number of continuous sleep fragments through the night), heart rate, blood glucose, activity (e.g., period of activity and/or the amount of calories burned from the activity at different periods of time), basal energy expenditure, weight, food (e.g., amount of calories consumed, dietary information), and/or static information about the user (e.g., height, waist circumference, age, diabetes type (if any)). To obtain the raw input data from the user, the system can use any of the user devices 104 described above in relation to FIG. 1. For example, the system can use blood pressure sensors to obtain blood pressure data, sleep trackers to obtain sleep data, heart rate sensors to obtain heart rate data, blood glucose sensors to obtain blood glucose data, motion sensors to obtain activity data, fitness trackers to obtain basal energy expenditure, and/or smart devices (e.g., smartphones, computers, tablet computers, smartwatches) to obtain user-inputted weight, food, or static information data about the user.

After obtaining the raw input data, at step 304, the system can generate model features to be subsequently used by one or more machine learning models. To generate the features, the system can transform the raw input data into structured matrices/vectors by grouping and/or aggregating the raw input data over time (the data can be timestamped) and across data input types. In some embodiments, the transformations used can depend on the type of data. For example, the system can convert static raw input data (e.g., data that does not change significantly in a short period of time, a predetermined period of time, etc.), such as gender, age, and diabetes type, into unordered categorical values (e.g., one-hot encoding, ordinal encoding, dummy variable encoding). Conversely, the system can transform non-static raw input data (e.g., user-specific aggregated information or other data that changes over time) by aggregating or otherwise combining the non-static raw input data over time and/or across their respective data input types. Examples of features generated from non-static raw input data can include:
glucose data (e.g., average and/or standard-deviation blood glucose levels)
sleep data (e.g., average number of hours of sleep in a period of time, such as at night in the past week or month, REM sleep data, sleep movement data, etc.)
bedtime data (e.g., average bedtime for a period of time, such as a recent number of day(s), recent week(s) or month(s))
physical activity (e.g., exercise schedules, calories burned, sum of physical activity reported for the user in a period of time such as the past 30 days, activity intensity, activity duration, etc.)
medication data (e.g., administration of medication, dosages, schedule, etc.)
insulin data (e.g., average insulin intake, rate of insulin dosages, etc.)
food data (e.g., average amount of carbohydrates consumed by the user on average per day or other period of time, maximum/minimum carbohydrates consumed for a period of time, eating intervals, etc.)
averaged blood pressure data (e.g., average systolic and/or diastolic blood pressure in the past 7 days, 30 days, or other period of time)
blood pressure values (e.g., maximal and minimal blood pressure values over a period of time, such as in the past 7 days, week(s), 30 days, and/or months)
rate of blood pressure change data (e.g., average rate of change of blood pressure over period of time, such as the last 2 days, 1 week, 30 days, month(s))
blood pressure trend tag from retrospective trend identification function(s)
proportion of blood pressure readings identified, such as hypertensive or hypotensive readings
average, minimal, and maximal heart rate in a period of time
heart rate data (e.g., average maximal daily heart rate, average minimal daily heart rate, etc.)
estimated daily basal energy expenditure in the past day(s), week(s) and/or month(s)
dietary data from food logged by the user, such as the consumed dietary cholesterol, sodium, and/or alcohol.

The system can identify non-static raw input data, aggregate the non-static raw input data over any time range (e.g., past number of hours, days, weeks, months) and is not limited to the time ranges described above. The system can also generate the features even when many of these data types are missing for the user by using default/values in-place of the missing data or performing interpolation to extrapolate the missing data.

To develop a training set, at step 306, the system can sample, for each individual in the input dataset, the raw input data for each selected time period (e.g., week(s), day(s), hour(s), month(s), year(s)). The system can also sample, for each user, average blood pressure values (systolic and/or diastolic) for each subsequent fixed/selected time period (e.g., the following week(s), day(s), month(s), year(s)). In other words, the system can sample raw input data and corresponding ground-truth average blood pressure values in a following future time horizon. For example, the system can sample over 150,000 weekly average blood pressure values and raw input data from more than 6000 individuals in the input dataset. After sampling, the system can generate a set of features for each of the sampled raw input datapoints using the transformations described above.

At step 308, the system can subsequently generate model inputs by creating one or more tuples of features generated from raw input data sampled at fixed/selected time periods and corresponding ground-truth average blood pressure values sampled in subsequent time periods. The blood pressure values can be time-stamped to generate an order list blood pressure values. For example, the system can generate the following model inputs: {features generated from raw input data sampled at month 1, average blood pressure value sampled a week after month 1}, {features generated from raw input data sampled at month 2, average blood pressure value sampled a week after month 2}, etc. In some embodiments, to develop the training set, the system can sample raw input data and average blood pressure data up to a specific date (e.g., sample 99,000 datapoints up until the date Jan. 1, 2020). In such instances, the system can subsequently develop a testing set with datapoints sampled after the specific date to test the trained model as described in detail below (e.g., sample over 57,600 datapoints after the date Jan. 1, 2020). The system can generate model inputs by creating one or more ordered or sequenced data sets. The data sets can be tuples inputted to models disclosed herein.

Upon the development of the training set, at step 310, the system can train one or more machine learning models on the training set to predict the future systolic or diastolic blood pressure averaged over a fixed/selected time period. In some embodiments, the system can train two models. The first model can be a regression model that forecasts weekly average systolic or diastolic blood pressure one week in advance (can also be other time periods or forecast horizons, e.g., the next/subsequent month(s), day(s), hour(s), year(s)). The regression model can be a supervised learning model, such as a gradient boosted trees regression model, or any suitable regression model that learns a mapping from the set of featured input datapoints to the desired average blood pressure value for a future time period/horizon.

The second model can be a classification model that predicts whether the blood pressure of the user will rise, fall, or remain steady over the next/subsequent week (can also be other time periods or forecast horizons, e.g., the next/subsequent month(s), day(s)). To determine the change in the blood pressure, the system can determine whether the average blood pressure of the following week falls above, below, or within a predefined margin of acceptable change from a reading of the average blood pressure in the previous week. In some embodiments, if the average blood pressure falls above the predefined margin, then the model can output a class label of, e.g., “rise”. If the average blood pressure falls below the predefined margin, then the model can output a class label of, e.g., “fall”. If the average blood pressure is within the predefined margin, then the model can output a class label of, e.g., “steady”. For both the first and second model, the system can also use any of the one or more machine learning models described above in relation to system 102 in FIG. 1. The system can train the first and/or the second model on raw input data and blood pressure data from several different individuals/users simultaneously, so that the experiences of some individuals/users will inform the predictions of other individuals/users having similar features. Accordingly, the system can make predictions even for users with few datapoints and with irregular or incomplete raw input data.

Following the training of the models, at step 312, the system can use/deploy the trained models to forecast the average blood-pressure in a time period/horizon starting at a prediction or deployment time. In some embodiments, a user account can obtain data from a linked additional device to retrieve additional health data from the additional device. If a newly available device is identified, the system can determine whether to retrieve health data from the available device. For example, system can't determine whether to obtain additional data based on the training of the machine learning model utilized for the forecast, predictions, conduct, etc. In some embodiments, the system can select a data set from newly available health data based on similar or matching health data being used in the machine learning model training. The system could then generate at least one forecast and/or prediction by inputting the health data (including the new health data, into the machine learning model). This allows a system to add or remove user devices and data sources to, for example, increase accuracy of generated output, utilize health data suitable for machine learning models and other algorithms disclosed here in, etc.

At step 314, the system can evaluate the performance of the models at prediction/evaluation time. The system can use evaluation metrics including accuracy, precision, or recall. Based on the evaluated accuracy, the system can adjust or fine-tune parameters of the model (e.g., modifying hyperparameters, changing features or raw input data used, adjusting the fixed/selected time periods or horizons, tuning the weights of the model, using a different model or set of models) to improve evaluation metrics.

In some embodiments system, the system can modify data utilized in the models based on the performance evaluations. For example, the system can add or remove data streams from user devices 104 to increase performance. If performance falls below this type of level, the user can be notified via user device 104, for example, adjust data collection settings, utilize new user devices, modify operation of user devices, etc. The system can also notify the user to provide validation blood pressures (e.g., blood pressures using a blood pressure cuff, physician administered blood pressure tests, etc.) to provide known blood pressures. The known blood pressures can be utilized to calibrate blood pressure detection algorithms of user devices (e.g., device 200 of FIG. 2) with blood pressure detection capability.

To evaluate the accuracy of the regression model, the system can use the trained regression model to predict the future blood pressure averages on test datasets. The test dataset can include raw input data and average blood pressure data set aside for testing and not used during training (e.g., data sampled at a time period after that of the training data). For example, the system can evaluate the accuracy of the model on a test dataset and determine the resulting mean absolute error to be 4.76 mmHG. The system can repeat the training and evaluation procedures using different dates as the cutoff time (a parameter of the model) between the training dataset and the testing dataset.

For evaluating the accuracy of the classification model, the system can use a receiver operating character (ROC) curve generated from iterations of training and testing the classification model on different predefined margins of acceptable change in blood pressure. As an example, the system can determine the following areas under the ROC curve (AUC): rising blood pressure, 87.9%; steady blood pressure, 77.5%; and falling blood pressure, 88.3%. The weighted AUC for all the observations in this example can be 82.3%, depending on the weights used.

In some embodiments, the system can evaluate the accuracy contributions, from the different features/data types, to the models. The system can separate the features into different groups or buckets, each containing features related to a particular signal or behavior. For example, the groups or buckets can be: blood pressure features, blood glucose features, carbohydrate consumption features, physical activity, user data (e.g., weight, height, ethnicity, gender, BMI, age, etc.), user history (e.g., medical condition, family medical history, etc.), and other features disclosed herein. The system can evaluate the accuracy contributions from one or more of the different features by training and testing the model on every combination of the groups/buckets (only including features in the combination, while removing the features that are not in the combination). For example, the system can test how the model performs when trained only on blood pressure features and determine the accuracy to be 0.702525, mean absolute error to be 4.768478, and the AUC to be 0.82017. As another example, the system can test how the model performs when trained only on blood pressure and non-blood pressure data, such as blood glucose features, and determine the accuracy to be 0.703478, mean absolute error to be 4.767569, and the AUC to be 0.821327. In some embodiments, the system can test how the model performs when trained on any combination of the model features described herein. Below are example combinations of groups/buckets that can include the blood pressure group/bucket:

| | | mean absolute error | |
|---|---|---|---|
| Included groups/buckets | accuracy | (MAE) | AUC |
| BP only | 0.702525 | 4.768478 | 0.82017 |
| BP, BG | 0.703478 | 4.767569 | 0.821327 |
| BP, carbs | 0.702712 | 4.768426 | 0.820269 |
| BP, activity | 0.702824 | 4.76812 | 0.820017 |
| BP, weight | 0.702582 | 4.765538 | 0.819789 |
| BP, other | 0.704244 | 4.758854 | 0.82163 |
| BP, BG, carbs | 0.703422 | 4.767482 | 0.821502 |
| BP, BG, activity | 0.702936 | 4.76711 | 0.821362 |
| BP, BG, weight | 0.703067 | 4.764997 | 0.821148 |
| BP, BG, other | 0.704692 | 4.757667 | 0.822198 |
| BP, carbs, activity | 0.702936 | 4.768045 | 0.820128 |
| BP, carbs, weight | 0.70288 | 4.765526 | 0.81995 |
| BP, carbs, other | 0.704487 | 4.759033 | 0.821738 |
| BP, activity, | 0.702507 | 4.765435 | 0.819829 |
| weight | | | |
| BP, activity, other | 0.704674 | 4.758831 | 0.821748 |
| BP, weight, other | 0.704431 | 4.756253 | 0.821341 |
| BP, BG, carbs, | 0.703198 | 4.766989 | 0.821505 |
| activity | | | |
| BP, BG, carbs, | 0.703217 | 4.764939 | 0.821349 |
| weight | | | |
| BP, BG, carbs, | 0.70486 | 4.757681 | 0.822368 |
| other | | | |
| BP, BG, activity, | 0.70331 | 4.764733 | 0.821322 |
| weight | | | |
| BP, BG, activity, | 0.705253 | 4.757546 | 0.822428 |
| other | | | |
| BP, BG, weight, | 0.704524 | 4.755369 | 0.822034 |
| other | | | |
| BP, carbs, activity, | 0.7026 | 4.765427 | 0.81998 |
| weight | | | |
| BP, carbs, activity, | 0.704991 | 4.758992 | 0.821858 |
| other | | | |
| BP, carbs, weight, | 0.704468 | 4.75644 | 0.821518 |
| other | | | |
| BP, activity, | 0.70445 | 4.756366 | 0.821589 |
| weight, other | | | |
| BP, BG, carbs, | 0.703235 | 4.76464 | 0.821508 |
| activity, weight | | | |
| BP, BG, carbs, | 0.705327 | 4.757513 | 0.822584 |
| activity, other | | | |
| BP, BG, carbs, | 0.704393 | 4.755387 | 0.822245 |
| weight, other | | | |
| BP, BG, activity, | 0.705178 | 4.75536 | 0.82237 |
| weight, other | | | |
| BP, carbs, activity, | 0.704767 | 4.756553 | 0.821746 |
| weight, other | | | |
| BP, BG, carbs, | 0.70501 | 4.755336 | 0.822576 |
| activity, weight, | | | |
| other (i.e., all data | | | |
| types) | | | |

In some embodiments, the groups (e.g., buckets of data) can include only non-blood pressure data. Blood pressure data can be used to identify group(s) of non-blood pressure data associated with blood pressure for a user or groups of users. The system can then predict blood pressures, at least in part, on the non-blood pressure data. The system can also use different groups/buckets over a period of time. For example, the blood pressure and a first set of features can be used during a first period of time (e.g., sleeping or resting) and blood pressure and a second set of features can be used during a second period of time (e.g., awake period of time, exercise period of time, etc.). The periods of time, sampling rates/schedules, types of combined data, groups of data, and/or other data relationships can be determined based on, for example, user data (e.g., body motion, sleep data, etc.), user-inputted data, etc.

FIG. 3B is a block diagram illustrating a process 350 for predicting blood pressure levels, in accordance with embodiments of the present technology. The system can link a user account(s) to a plurality of devices (e.g., blood pressure sensors, blood pressure measuring cuffs, blood glucose sensors, pressure sensors, heart rate sensors, sleep trackers, temperature sensors, motion sensors, smartphone, tablet computer, smartwatch, fitness tracker, wearable blood pressure monitor, or other biomonitoring devices) of the user which send health data associated with the user.

At step 352, the system can obtain multiple data types of health data of a user, such as blood pressure data, sleep data, blood glucose data, user activity data, and/or dietary data. The system can retrieve the health data from the devices, or the user can send the health data to the system via the user interface. Each device can be programmed to collect the health data for predicting blood pressure and include a analyte-detecting microneedle array(s) configured to generate analyte data of the health data when the analyte-detecting microneedle array(s) extends into the user's skin. Each device can include non-invasive blood pressure sensor configured to generate blood pressure data of the health data. The system can identify a training set(s) with types of sensor data matching the obtained health data and train a machine-learning model using the identified training set(s) (as described in step 310 of FIG. 3A).

At step 354, the system can select health data from the obtained health data based on training of the machine-learning model trained on different data types training sets related to blood pressure. The system can select multiple types of available health data (e.g., blood pressure data, sleep data, blood glucose data, user activity data, and/or dietary data) of the user and select data from each of the multiple types. The system can input the selected data into the trained machine-learning model to generate a blood pressure forecast and/or blood pressure prediction. In some cases, the selected multiple types of available health data of the user are a subset of all of the multiple types of available health data based on training data used to train the machine learning model. In some cases, a data type of the data set matches a data type of training sets used to training the machine-learning model.

At step 356, the system inputs the selected health data into the machine-learning model to determine a future blood pressure value (e.g., the blood pressure forecast) the blood pressure event prediction, and/or a contributing health factor(s). The system can generate a blood pressure forecast by inputting the health data from the wearable biosensor device and data set into the at least one machine-learning model. At step 358, the system can determine whether the future blood pressure value exceeds a threshold (an acceptable blood pressure range or hypotension value) or is below the threshold.

In response to the future blood pressure value exceeding a threshold, at step 360, the system sends a notification to the user interface regarding the future blood pressure value. The system can send executable instructions to the health data collecting devices to adjust operation of the devices based on the blood pressure forecast, the blood pressure event prediction, and/or a contributing health factor(s). The system can determine blood pressure monitoring settings for the devices based on output from the machine-learning model to achieve a threshold confidence score for additional predictions and transmit the blood pressure monitoring settings to the devices. The system can evaluate predictions based on the transmitted blood pressure monitoring settings used by the health monitoring devices.

FIG. 4A-4B illustrate examples of an interface displaying prompts (FIG. 4A) and reinforcements (FIG. 4B) output on a user devices 402 and 412 by the blood pressure prediction system configured in accordance with embodiments of the present technology. The reinforcements can include pressure data, progress to goal data, analytics, or other information disclosed herein. The interface of FIGS. 4A and 4B can be touch screens, electronic displays, and other interfaces disclosed herein. The prompts and reinforcements can be displayed on the interface 224 of FIG. 2. Machine-learning modules can be used to determine the prompts, reinforcements, and/or data suitable for the user.

### Additional Embodiments

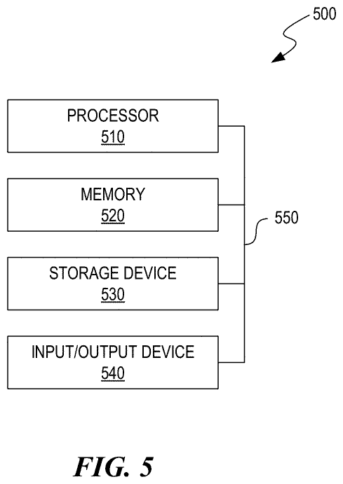

FIG. 5 is a schematic block diagram of a computing system or device (“system 500”) configured in accordance with embodiments of the present technology. The system 500 can be incorporated into or used with any of the systems and devices described herein, such as the system 102 of FIG. 1, user devices 104 of FIG. 1, user devices 200 of FIG. 2. The system 500 can be used to perform any of the processes or methods described herein with respect to FIGS. 1 and 2. The system 500 can include a processor 510, a memory 520, a storage device 530, and an input/output device 540. Each of the components 510, 520, 530 and 540 can be interconnected using a system bus 550. The processor 510 can be configured to process instructions for execution within the system 500. In some embodiments, the processor 510 can be a single-threaded processor. In alternate embodiments, the processor 510 can be a multi-threaded processor. Although FIG. 5 illustrates a single processor 510, in other embodiments the system 500 can include multiple processors 510. In such embodiments, some or all of the processors 510 can be situated at different locations. For example, a first processor can be located in a sensor device, a second processor can be located in a user device (e.g., a mobile device), and/or a third processor can be part of a cloud computing system or device.

The processor 510 can be further configured to process instructions stored in the memory 520 or on the storage device 530, including receiving or sending information through the input/output device 540. The memory 520 can store information within the system 500. In some embodiments, the memory 520 can be a computer-readable medium. In alternate embodiments, the memory 520 can be a volatile memory unit. In yet some embodiments, the memory 520 can be a non-volatile memory unit. The storage device 530 can be capable of providing mass storage for the system 500. In some embodiments, the storage device 530 can be a computer-readable medium. In alternate embodiments, the storage device 530 can be a floppy disk device, a hard disk device, an optical disk device, a tape device, non-volatile solid-state memory, or any other type of storage device. The input/output device 540 can be configured to provide input/output operations for the system 500. In some embodiments, the input/output device 540 can include a keyboard and/or pointing device. In alternate embodiments, the input/output device 540 can include a display unit for displaying graphical user interfaces.

Non-transitory computer program products (i.e., physically embodied computer program products) are also described that store instructions that, when executed by one or more data processors of one or more computing systems, cause at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including but not limited to a connection over a network (e.g., the Internet, a wireless wide area network, a local area network, a wide area network, a wired network, or the like), via a direct connection between one or more of the multiple computing systems, etc.

The systems and methods disclosed herein can be embodied in various forms including, for example, a data processor, such as a computer that also includes a database, digital electronic circuitry, firmware, software, or in combinations of them. Moreover, the above-noted features and other aspects and principles of the present disclosed implementations can be implemented in various environments. Such environments and related applications can be specially constructed for performing the various processes and operations according to the disclosed implementations or they can include a general-purpose computer or computing platform selectively activated or reconfigured by code to provide the necessary functionality. The processes disclosed herein are not inherently related to any particular computer, network, architecture, environment, or other apparatus, and can be implemented by a suitable combination of hardware, software, and/or firmware. For example, various general-purpose machines can be used with programs written in accordance with teachings of the disclosed implementations, or it can be more convenient to construct a specialized apparatus or system to perform the required methods and techniques.

The systems and methods disclosed herein can be implemented as a computer program product, i.e., a computer program tangibly embodied in an information carrier, e.g., in a machine readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

These computer programs, which can also be referred to programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random-access memory associated with one or more physical processor cores.

To provide for interaction with a user, the subject matter described herein can be implemented on a computer having a display device, such as for example a cathode ray tube (CRT) or a liquid crystal display (LCD) monitor for displaying information to the user and a keyboard and a pointing device, such as for example a mouse or a trackball, by which the user can provide input to the computer. Alternatively, or in combination, the display device can be a touchscreen or other user input device configured to accept tactile input (e.g., via a virtual keyboard and mouse). Other kinds of devices can be used to provide for interaction with a user as well. For example, feedback provided to the user can be any form of sensory feedback, such as for example visual feedback, auditory feedback, or tactile feedback; and input from the user can be received in any form, including, but not limited to, acoustic, speech, or tactile input.

The technology described herein can be implemented in a computing system that includes a back-end component, such as for example one or more data servers, or that includes a middleware component, such as for example one or more application servers, or that includes a front-end component, such as for example one or more client computers having a graphical user interface or a Web browser through which a user can interact with an implementation of the subject matter described herein, or any combination of such back-end, middleware, or front-end components. The components of the system can be interconnected by any form or medium of digital data communication, such as for example a communication network. Examples of communication networks include, but are not limited to, a local area network (“LAN”), a wide area network (“WAN”), and the Internet.

The computing system can include clients and servers. A client and server are generally, but not exclusively, remote from each other and typically interact through a communication network. The relationship of client and server arises by virtue of computer programs running on the respective computers and having a client-server relationship to each other.

### EXAMPLES

The present technology is illustrated, for example, according to various aspects described below. Various examples of aspects of the present technology are described as numbered examples (1, 2, 3, etc.) for convenience. These are provided as examples and do not limit the present technology. It is noted that any of the dependent examples can be combined in any suitable manner, and placed into a respective independent example. The other examples can be presented in a similar manner.

1. A computer-implemented method for a wearable biosensor device-based blood pressure prediction of a user, the computer-implemented method comprising:
receiving, from a wearable biosensor device, health data of the user, wherein the wearable biosensor device is programmed to collect the health data for predicting blood pressure and includes
   at least one analyte-detecting microneedle array configured to generate analyte data of the health data when the at least one analyte-detecting microneedle array extends into skin of the user, and
   a non-invasive blood pressure sensor configured to generate blood pressure data of the health data;
generating features for at least one machine-learning model by transforming the health data into vectors;
generating one or more model inputs by creating tuples of the features sampled at selected time periods and corresponding blood pressure values sampled in subsequent time periods;
training at least one machine-learning model to:
   determine a forecast of a blood pressure value (e.g., an average blood pressure value, a activity blood pressure value, a daytime blood pressure average, a nighttime blood pressure average, a weekly blood pressure average, etc.) of the user over a time period; and
   determine a blood pressure event prediction during the time period;
outputting, to an electronic user interface, a notification including the forecast, the prediction, and one or more contributing health factors.
   2. The computer-implemented method of example 1, further comprising sending executable instructions to the wearable biosensor device to adjust operation of the wearable biosensor device based on at least one of the forecast, the blood pressure event prediction, or one or more contributing health factors.
   3. The computer-implemented method of any of examples 1-2, further comprising:
determining blood pressure monitoring settings for the wearable biosensor device based on output from the at least one machine-learning model to achieve a threshold confidence score for additional predictions;
transmitting the blood pressure monitoring settings to the wearable biosensor device; and
evaluating predictions based on the transmitted blood pressure monitoring settings used by the wearable biosensor device.
   4. The computer-implemented method of any of examples 1-3, further comprising:
identifying one or more training sets with types of sensor data matching the health data; and
training the at least one machine-learning model using the identified one or more training sets.
   5. The computer-implemented method of any of examples 1-4, further comprising:
selecting multiple types of available health data of the user;
selecting data from each of the multiple types of available health data of the user; and
inputting the selected data into the trained at least one machine-learning model to generate at least one of a blood pressure forecast or a blood pressure prediction.
   6. The computer-implemented method of any of examples 1-5, wherein the types of available health data include:
blood pressure data,
sleep data,
blood glucose data,
user activity data, and/or
dietary data.
   7. The computer-implemented method of any of examples 1-6, wherein the selected multiple types of available health data of the user are a subset of all of the multiple types of available health data, wherein the selection of the subset of types of available health data is based on training data used to train the at least one machine learning model.
   8. The computer-implemented method of any of examples 1-7, further comprising:
linking a user account with at least one additional device;
retrieving additional health data from each of the at least one additional device;
selecting a data set from the additional health data based on the training of the at least one machine-learning model;
generating at least one of the forecast or the prediction by inputting the health data from the wearable biosensor device and data set into the at least one machine-learning model.
   9. The computer-implemented method of any of examples 1-8, wherein a data type of the data set matches a data type of training sets used to training the at least one machine-learning model.
   10. The computer-implemented method of any of examples 1-9, wherein the blood pressure event prediction is whether the blood pressure of the user will be one or more of
outside of an acceptable blood pressure range,
below a threshold hypotension value,
above a threshold hypertension value.
   11. The computer-implemented method of any of examples 1-10, wherein the blood pressure event prediction is a target blood pressure value associated with implementation of the one or more contributing health factors.
   12. The computer-implemented method of any of examples 1-11, wherein the target blood pressure value is at least one of a target systolic blood pressure value, a target diastolic blood pressure value, or a target change of blood pressure.
   13. The computer-implemented method of any of examples 1-12, wherein determining the blood pressure event prediction during the time period, further comprises:
determining whether the average blood pressure during the time period falls above, below, or within a predefined margin of acceptable change from a reading of the average blood pressure in a previous time period, wherein the previous time period and the time period are a same length.
   14. The computer-implemented method of any of examples 1-13, wherein generating the features for the at least one machine-learning model by transforming the health data into the vectors, further comprises:
grouping or aggregating the health data over the time period and across data input types.
   15. The computer-implemented method of any of examples 1-15, wherein outputting, to the user interface, the notification for the user, further comprises:
recommending self-care to the user to decrease the blood pressure in a future time period, wherein the self-care includes sleep habits, diet routines, or exercise routines for the user.
   16. The computer-implemented method of any of examples 1-15, wherein:
the health data includes one or more of blood pressure data, blood glucose data, heart rate data, food data, activity data, sleep data, weight data, medication data, diagnosis data, or demographics data; and
the one or more contributing health factors include one or more of blood pressure, blood glucose, heart rate, food, activity, sleep, weight, medication, diagnosis, or demographics.
   17. The computer-implemented method of any of examples 1-16, further comprising:
analyzing the health data to identify a first subset of data related to a first set of blood pressure measurements during a first period and second subset of data related to a second set of blood pressure measurements during a second period,
   wherein during the first period the user is awake, and wherein during the second period the user is asleep.
      18. The computer-implemented method of any of examples 1-17,
collecting, from the wearable biosensor device, additional health data of the user;
determining the blood pressure of the user changed outside of a predefined margin during the time period; and
outputting, to the user interface, a second notification for the user indicating the blood pressure changed outside of the predefined margin.
   19. A system comprising:
one or more processors; and
one or more memories storing instructions that, when executed by the one or more processors, cause the system to perform a process for a wearable biosensor device-based blood pressure prediction of a user, the process comprising:
   receiving, from a wearable biosensor device, health data of the user, wherein the wearable biosensor device is programmed to collect the health data for predicting blood pressure and includes
      at least one analyte-detecting microneedle array configured to generate analyte data of the health data when the at least one analyte-detecting microneedle array extends into skin of the user, and
      a non-invasive blood pressure sensor configured to generate blood pressure data of the health data;
   generating features for at least one machine-learning model by transforming the health data into vectors;
   generating one or more model inputs by creating tuples of the features sampled at selected time periods and corresponding blood pressure values sampled in subsequent time periods;
   training at least one machine-learning model to:
      determine a forecast of a blood pressure value (e.g., an average blood pressure value, a medium blood pressure value, a daytime blood pressure average, a nighttime blood pressure average, a weekly blood pressure average, etc.) of the user over a time period; and
      determine a blood pressure event prediction during the time period;
   outputting, to an electronic user interface, a notification including the forecast, the prediction, and one or more contributing health factors.
      20. The system according to example 19, wherein determining the blood pressure event prediction during the time period, further comprises:
determining whether the average blood pressure during the time period falls above, below, or within a predefined margin of acceptable change from a reading of the average blood pressure in a previous time period, wherein the previous time period and the time period are a same length.
   21. The system according to any of examples 19-20, wherein generating the features for the at least one machine-learning model by transforming the health data into the vectors, further comprises:
grouping or aggregating the health data over the time period and across data input types.
   22. The system according to any of examples 19-21, wherein outputting, to the user interface, the notification for the user, further comprises:
recommending self-care to the user to decrease the blood pressure in a future time period, wherein the self-care includes sleep habits, diet routines, or exercise routines for the user.
   23. The system according to any of examples 19-22, wherein:
the health data includes one or more of blood pressure data, blood glucose data, heart rate data, food data, activity data, sleep data, weight data, medication data, diagnosis data, or demographics data; and
the one or more contributing health factors include one or more of blood pressure, blood glucose, heart rate, food, activity, sleep, weight, medication, diagnosis, or demographics.
   24. The system according to any of examples 19-23, wherein the process further comprises:
analyzing the health data to identify a first subset of data related to a first set of blood pressure measurements during a first period and second subset of data related to a second set of blood pressure measurements during a second period,
   wherein during the first period the user is awake, and wherein during the second period the user is asleep.
      25. The system according to any of examples 19-24, wherein the process further comprises:
collecting, from the wearable biosensor device, additional health data of the user;
determining the blood pressure of the user changed outside of a predefined margin during the time period; and
outputting, to the user interface, a second notification for the user indicating the blood pressure changed outside of the predefined margin.
   26. A non-transitory computer-readable medium storing instructions that, when executed by a computing system, cause the computing system to perform operations for a wearable biosensor device-based blood pressure prediction of a user, the operations comprising:
receiving, from a wearable biosensor device, health data of the user, wherein the wearable biosensor device is programmed to collect the health data for predicting blood pressure and includes
   at least one analyte-detecting microneedle array configured to generate analyte data of the health data when the at least one analyte-detecting microneedle array extends into skin of the user, and
   a non-invasive blood pressure sensor configured to generate blood pressure data of the health data;
generating features for at least one machine-learning model by transforming the health data into vectors;
generating one or more model inputs by creating tuples of the features sampled at selected time periods and corresponding blood pressure values sampled in subsequent time periods;
training at least one machine-learning model to:
   determine a forecast of a blood pressure value (e.g., an average blood pressure value, a medium blood pressure value, a daytime blood pressure average, a nighttime blood pressure average, a weekly blood pressure average, etc.) of the user over a time period; and
determine a blood pressure event prediction during the time period;
outputting, to an electronic user interface, a notification including the forecast, the prediction, and one or more contributing health factors.
   27. The non-transitory computer-readable medium of example 26, determining the blood pressure event prediction during the time period, further comprises:
determining whether the average blood pressure during the time period falls above, below, or within a predefined margin of acceptable change from a reading of the average blood pressure in a previous time period, wherein the previous time period and the time period are a same length.
   28. The non-transitory computer-readable medium of any of examples 26-27, wherein generating the features for the at least one machine-learning model by transforming the health data into the vectors, further comprises:
grouping or aggregating the health data over the time period and across data input types.
   29. The non-transitory computer-readable medium of any of examples 26-28, wherein outputting, to the user interface, the notification for the user, further comprises:
recommending self-care to the user to decrease the blood pressure in a future time period, wherein the self-care includes sleep habits, diet routines, or exercise routines for the user.
   30. The non-transitory computer-readable medium of any of examples 26-29, wherein:
the health data includes one or more of blood pressure data, blood glucose data, heart rate data, food data, activity data, sleep data, weight data, medication data, diagnosis data, or demographics data; and
the one or more contributing health factors include one or more of blood pressure, blood glucose, heart rate, food, activity, sleep, weight, medication, diagnosis, or demographics.
   31. The non-transitory computer-readable medium of any of examples 26-30, wherein the operations further comprise:
analyzing the health data to identify a first subset of data related to a first set of blood pressure measurements during a first period and second subset of data related to a second set of blood pressure measurements during a second period,
   wherein during the first period the user is awake, and wherein during the second period the user is asleep.
      32. A computer-implemented method for predicting blood pressure levels of a user, the computer-implemented method comprising:
obtaining multiple data types of health data of the user;
selecting health data from the obtained health data based on training of at least one machine-learning model trained on different data types training sets related to blood pressure;
inputting the selected health data into the at least one machine-learning model to determine a future blood pressure value; and
in response to the future blood pressure value exceeding a threshold, causing a notification to be sent to the user.
   33. The computer-implemented method of example 32, further comprising:
linking a user account to a plurality of devices of the user, wherein each of the plurality of devices is configured to send health data of the user;
   34. The computer-implemented method of any of examples 32-33, wherein the multiple data types of health data include:
blood pressure data,
sleep data,
blood glucose data,
user activity data, and/or
dietary data.
   35. The computer-implemented method of any of examples 32-34, further comprising:
identifying one or more training sets with types of sensor data matching the obtained health data; and
training the at least one machine-learning model using the identified one or more training sets.
   36. The computer-implemented method of any of examples 32-35, further comprising:
selecting multiple types of available health data of the user;
selecting data from each of the multiple types of available health data of the user; and
inputting the selected data into the trained at least one machine-learning model to generate at least one of a blood pressure forecast or blood pressure prediction.
   37. The computer-implemented method of any of examples 32-36, wherein the types of available health data include:
blood pressure data,
sleep data,
blood glucose data,
user activity data, and/or
dietary data.
   38. The computer-implemented method of any of examples 32-37, wherein the selected multiple types of available health data of the user are a subset of all of the multiple types of available health data, wherein the selection of the subset of types of available health data is based on training data used to train the at least one machine learning model.
   39. The computer-implemented method of any of examples 32-38, further comprising:
linking a user account with at least one additional device;
retrieving additional health data from each of the at least one additional device;
selecting a data set from the additional health data based on the training of the at least one machine-learning model;
generating at least one of a blood pressure forecast by inputting the health data from the at least one additional device and data set into the at least one machine-learning model.
   40. The computer-implemented method of any of examples 32-39, wherein a data type of the data set matches a data type of training sets used to training the at least one machine-learning model.

The embodiments set forth in the foregoing description do not represent all embodiments consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the embodiments described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other embodiments can be within the scope of the following claims.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise.

As used herein, the phrase “and/or” as in “A and/or B” refers to A alone, B alone, and A and B.

As used herein, the term “user” can refer to any entity including a person or a computer.

Although ordinal numbers such as first, second, and the like can, in some situations, relate to an order; as used in this document ordinal numbers do not necessarily imply an order. For example, ordinal numbers can be merely used to distinguish one item from another. For example, to distinguish a first event from a second event, but need not imply any chronological ordering or a fixed reference system (such that a first event in one paragraph of the description can be different from a first event in another paragraph of the description).

Furthermore, the skilled artisan will recognize the interchangeability of various features from different embodiments disclosed herein and disclosed in U.S. Pat. Nos. 9,008,745; 9,182,368; 10,173,042; U.S. application Ser. No. 15/601,204 (US Pub. No. 2017/0251958); U.S. application Ser. No. 15/876,678 (U.S. Pub. No. 2018/0140235); U.S. App. No. 63/034,331; U.S. App. No. 63/034,333; U.S. application Ser. No. 14/812,288 (US Pub. No. 2016/0029931); U.S. application Ser. No. 14/812,288 (US Pub. No. 2016/0029966); US Pub. No. 2017/0128009; U.S. application Ser. No. 17/236,753; PCT Application No.: PCT/2021/028445; U.S. App. No. 62/855,194; U.S. App. No. 62/854,088; U.S. App. No. 62/970,282; and PCT App. No. PCT/US19/49270 (WO2020/051101). For example, methods of detection, sensors, detection elements, biosensors, user devices, etc. can be incorporated into or used with the technology disclosed herein. All of these applications are incorporated herein by reference in their entireties. Similarly, the various features and acts discussed above, as well as other known equivalents for each such feature or act, can be mixed and matched by one of ordinary skill in this art to perform methods in accordance with principles described herein. All of the above cited applications and patents are herein incorporated by reference in their entireties.

From the foregoing, it will be appreciated that specific embodiments of the invention have been described herein for purposes of illustration, but that various modifications may be made without deviating from the scope of the invention. Accordingly, the invention is not limited except as by the appended claims.

1. A computer-implemented method for a wearable biosensor device-based blood pressure prediction of a user, the computer-implemented method comprising:
receiving, from a wearable biosensor device, health data of the user, wherein the wearable biosensor device is programmed to collect the health data for predicting blood pressure and includes
at least one analyte-detecting microneedle array configured to generate analyte data of the health data when the at least one analyte-detecting microneedle array extends into skin of the user, and
a non-invasive blood pressure sensor configured to generate blood pressure data of the health data;
generating features for at least one machine-learning model by transforming the health data into vectors;
generating one or more model inputs by creating tuples of the features sampled at selected time periods and corresponding blood pressure values sampled in subsequent time periods;
training at least one machine-learning model to:
determine a forecast of an average blood pressure of the user over a time period; and
determine a blood pressure event prediction during the time period;
outputting, to an electronic user interface, a notification including the forecast, the prediction, and one or more contributing health factors.

2. The computer-implemented method of claim 1, further comprising sending executable instructions to the wearable biosensor device to adjust operation of the wearable biosensor device based on at least one of the forecast, the blood pressure event prediction, or one or more contributing health factors.

3. The computer-implemented method of claim 1, further comprising:
determining blood pressure monitoring settings for the wearable biosensor device based on output from the at least one machine-learning model to achieve a threshold confidence score for additional predictions;
transmitting the blood pressure monitoring settings to the wearable biosensor device; and
evaluating predictions based on the transmitted blood pressure monitoring settings used by the wearable biosensor device.

4. The computer-implemented method of claim 1, further comprising:
identifying one or more training sets with types of sensor data matching the health data; and
training the at least one machine-learning model using the identified one or more training sets.

5. The computer-implemented method of claim 1, further comprising:
selecting multiple types of available health data of the user;
selecting data from each of the multiple types of available health data of the user; and
inputting the selected data into the trained at least one machine-learning model to generate at least one of a blood pressure forecast or a blood pressure prediction.

6. The computer-implemented method of claim 5, wherein the types of available health data include:
blood pressure data,
sleep data,
blood glucose data,
user activity data, and/or
dietary data.

7. The computer-implemented method of claim 5, wherein the selected multiple types of available health data of the user are a subset of all of the multiple types of available health data, wherein the selection of the subset of types of available health data is based on training data used to train the at least one machine learning model.

8. The computer-implemented method of claim 1, further comprising:
linking a user account with at least one additional device;
retrieving additional health data from each of the at least one additional device;
selecting a data set from the additional health data based on the training of the at least one machine-learning model; and
generating at least one of the forecast or the prediction by inputting the health data from the wearable biosensor device and the data set into the at least one machine-learning model.

9. The computer-implemented method of claim 8, wherein a data type of the data set matches a data type of training sets used to training the at least one machine-learning model.

10. The computer-implemented method of claim 1, wherein the blood pressure event prediction is whether the blood pressure of the user will be one or more of
outside of an acceptable blood pressure range,
below a threshold hypotension value, and
above a threshold hypertension value.

11. The computer-implemented method of claim 1, wherein the blood pressure event prediction is a target blood pressure value associated with implementation of the one or more contributing health factors.

12. The computer-implemented method of claim 11, wherein the target blood pressure value is at least one of a target systolic blood pressure value, a target diastolic blood pressure value, or a target change of blood pressure.

13. The computer-implemented method of claim 1, wherein determining the blood pressure event prediction during the time period, further comprises:
determining whether the average blood pressure during the time period falls above, below, or within a predefined margin of acceptable change from a reading of the average blood pressure in a previous time period, wherein the previous time period and the time period are a same length.

14. The computer-implemented method of claim 1, wherein generating the features for the at least one machine-learning model by transforming the health data into the vectors, further comprises:
grouping or aggregating the health data over the time period and across data input types.

15. The computer-implemented method of claim 1, wherein outputting, to the user interface, the notification for the user, further comprises:
recommending self-care to the user to decrease the blood pressure in a future time period, wherein the self-care includes one or more or sleep habits, diet routines, and/or exercise routines for the user.

16. The computer-implemented method of claim 1, wherein:
the health data includes one or more of blood pressure data, blood glucose data, heart rate data, food data, activity data, sleep data, weight data, medication data, diagnosis data, or demographics data; and
the one or more contributing health factors include one or more of blood pressure, blood glucose, heart rate, food, activity, sleep, weight, medication, diagnosis, or demographics.

17. The computer-implemented method of claim 1, further comprising:
analyzing the health data to identify a first subset of data related to a first set of blood pressure measurements during a first period and second subset of data related to a second set of blood pressure measurements during a second period,
wherein during the first period the user is awake, and wherein during the second period the user is asleep.

18. The computer-implemented method of claim 1,
collecting, from the wearable biosensor device, additional health data of the user;
determining the blood pressure of the user changed outside of a predefined margin during the time period; and
outputting, to the user interface, a second notification for the user indicating the blood pressure changed outside of the predefined margin.

19. A system comprising:
one or more processors; and
one or more memories storing instructions that, when executed by the one or more processors, cause the system to perform a process for a wearable biosensor device-based blood pressure prediction of a user, the process comprising:
receiving, from a wearable biosensor device, health data of the user, wherein the wearable biosensor device is programmed to collect the health data for predicting blood pressure and includes
at least one analyte-detecting microneedle array configured to generate analyte data of the health data when the at least one analyte-detecting microneedle array extends into skin of the user, and
a non-invasive blood pressure sensor configured to generate blood pressure data of the health data;
generating features for at least one machine-learning model by transforming the health data into vectors;
generating one or more model inputs by creating tuples of the features sampled at selected time periods and corresponding blood pressure values sampled in subsequent time periods;
training at least one machine-learning model to:
determine a forecast of an average blood pressure of the user over a time period; and
determine a blood pressure event prediction during the time period;
outputting, to an electronic user interface, a notification including the forecast, the prediction, and one or more contributing health factors.

20.-40. (canceled)
